(19) United States
(12) Patent Application Publication
(10) Pub. No.: US 2010/0152864 A1
(43) Pub. Date: Jun. 17, 2010

(54) OSSEOINTEGRATED IMPLANT WITH ELECTRICAL STIMULATION

(75) Inventors: Brad M. Isaacson, Salt Lake City, UT (US); Roy D. Bloebaum, Salt Lake City, UT (US); Dustin L. Williams, Bountiful, UT (US)

Correspondence Address: KELLY LOWRY & KELLEY, LLP, 6320 CANOGA AVENUE, SUITE 1650, WOODLAND HILLS, CA, 91367, US

(73) Assignees: UNIVERSITY OF UTAH, Salt Lake City, UT (US); U.S. DEPARTMENT OF VETERANS AFFAIRS, Washington, DC (US)

(21) Appl. No.: 12/576,549

(22) Filed: Oct. 9, 2009

Related U.S. Application Data
(60) Provisional application No. 61/201,778, filed on Dec. 15, 2008.

(51) Int. Cl. A61F 2/74; A61N 1/00

(52) U.S. Cl. 623/27; 607/48

(57) ABSTRACT

An osseointegrated implant for an amputee includes a surgically implanted post-shaped orthopedic implant rigidly supported by patient bone, wherein the implant protrudes through overlying stump tissue to an external location for suitable attachment to a prosthetic limb or the like. A non-invasive electrical stimulation system provides controlled electrical stimulation at the implant-bone interface for achieving rapid and secure fixation by osseointegration in a significantly reduced rehabilitation time. The electrical stimulation system utilizes at least one and preferably multiple electrodes mounted externally onto the patient in close proximity to the implant-bone interface, with an external portion of the implant providing a second electrode. In one preferred form, the implant comprises a base structure of titanium or titanium alloy or the like, coated with a thin film of a highly conductive substance such as gold for improved electrical conductivity.

This invention was made with government support under W81XWH-06-1-0574 awarded by ARMY/MRMC—Medical Research and Material Command. The government has certain rights in the invention.

### BACKGROUND OF THE INVENTION

This invention was made with government support by the U.S. Government as represented by the Department of Veterans Affairs. The government has certain rights in the invention.

This invention relates generally to improvements in external or exoskeletal prosthetic devices and systems of the type utilizing an implanted, bone anchored post-shaped orthopedic implant having or carrying an externally protruding or externally exposed fixator structure for removable attachment to a prosthesis such as a prosthetic limb or the like. More particularly, this invention relates to an improved attachment system utilizing an electrical stimulation system for expediting skeletal fixation by osseointegration at an interface between the bone anchored mounting post and patient bone. As a result, significant reductions in patient rehabilitation times are achieved.

In the past, limb amputations have traditionally provided a soft tissue interface at the end of an amputated limb such as a leg or arm, wherein this soft tissue interface overlies the end of the amputated or residual patient bone. Socket type prosthetic limbs such as prosthetic leg and arm structures have been generally known in the art, wherein a prosthesis is constructed with an open-ended and typically padded socket structure for receiving and supporting the post-surgical stump of an amputated limb. By way of one example, a typical socket type prosthetic leg includes such open-ended socket structure at an upper end thereof for receiving and supporting the post-surgical residual upper leg, e.g. of a transfemoral amputee. Various straps and/or other fasteners are provided for securing the prosthetic leg to the amputated limb to accommodate walking mobility at least on a limited basis. Such prosthetic limbs can be an important factor in both physical and mental rehabilitation of an amputee.

However, socket type prosthetic limbs are associated with a number of recognized limitations and disadvantages. In particular, the socket style prosthesis inherently couples mechanical loads associated with normal ambulatory activity through the soft tissue interface defined by the soft tissue covering the end or stump of the amputated limb, but wherein this soft tissue interface is structurally unsuited for this purpose. While many different arrangements and configurations for the requisite straps and other fasteners have been proposed for improved transmission and distribution of these mechanical loads to bone structures to achieve an improved secure and stable prosthesis attachment, to correspondingly accommodate a more natural ambulatory movement, such arrangements have achieved only limited success. In addition, compressive loading of the soft stump tissue interface often results in blisters, sores, chafing and other undesirable skin irritation problems which have been addressed primarily by adding soft padding material within the socket structure. But such soft padding material undesirably increases the extent of the soft or non-rigid interface between the amputated limb and prosthesis, all in a manner that is incompatible with an optimally secure and stable prosthesis connection. As a result, particularly in the case of a prosthetic leg, traditional socket style connection structures and methods have generally failed to accommodate a normal walking motion.

Problems associated with compression of the soft stump tissue within and/or against a conventional socket type prosthetic limb are compounded by heterotopic ossification (HO) which comprises bizarre overgrowth of mature osseous bone into the neighboring soft tissue. Such HO thus essentially comprises relatively hard and sharp bone tissue which grows into the adjacent soft tissue, such as the stump tissue, resulting in potentially significant amputee discomfort when such sharp osseous formations are compressed within and/or against the soft tissue while using a socket type prosthetic limb. HO has been a particular problem encountered by military veterans subjected to amputation by injuries related to improvised explosive devices, wherein such veterans are otherwise relatively young, in good physical condition, and desire a relatively active lifestyle.

In recent years, improved external or exoskeletal prosthetic devices have been proposed, wherein the external prosthesis is structurally linked by means of a bone anchored mounting system including a post-shaped orthopedic implant securely attached directly to patient bone. In such devices, a rigid mounting post-shaped implant is surgically implanted and attached securely to patient bone as by means of osseointegration or the like. This implant extends from the bone attachment site and includes or is attached to a fixator pin or post structure that protrudes percutaneously through the overlying soft stump tissue at the end of the amputated limb. Thus, one end of the orthopedic implant is externally exposed for secure and direct mechanical attachment to a prosthetic limb or the like by means of a rigid linkage. See, e.g., U.S. 2008/0288087 and U.S. 2009/0005820, which are incorporated by reference herein.

In such bone anchored mounting systems, mechanical loads on the prosthetic limb during ambulation are thus transmitted by the rigid linkage and through the external fixator structure and orthopedic implant directly to patient bone. As a result, conventional and undesirable mechanical loading of the soft tissue interface is avoided, and substantially improved and/or substantially normal patient movements are accommodated. In addition, the requirement for compressive loading of the soft tissue at the end of the amputated limb is substantially eliminated, to correspondingly reduce incidence of blisters and other associated skin irritation problems. Compression of the stump tissue overlying the end of the amputated patient bone is beneficially eliminated. Moreover, by mechanically linking and supporting the prosthesis directly from patient bone, amputees have reported a significant increase in perception of the prosthesis as an actual and natural body part—a highly desirable factor referred to as “osseoperception”.

While use of an osseointegrated bone anchored mounting system offers potentially dramatic improvements in amputee lifestyle, secure and stable osseointegration attachment of the bone anchored implant is a slow biological process typically requiring from several months up to about 2 years before substantially full attachment strength at the implant-bone interface is achieved. For many amputees, and especially for leg amputees, achievement of substantially full attachment strength at the implant-bone interface is required before the implant can be subjected to mechanical loads of the type encountered during normal walking and the like. Unfortunately, the rehabilitation or recovery time needed for substantially full strength attachment of an osseointegrated bone anchored implant can be too long for many amputees.

There exists, therefore, a significant need for further improvements in and to osseointegrated implant systems particularly of the type using a bone anchored orthopedic implant for external attachment to a prosthetic limb or the like, wherein substantially full strength attachment of the bone anchored implant can be achieved in a significantly reduced period of rehabilitation time. By using controlled electrical stimulation, the present invention fulfills these needs and is believed to provide further related advantages.

### SUMMARY OF THE INVENTION

In accordance with the invention, an osseointegrated implant includes a surgically implanted post-shaped orthopedic prosthesis or implant rigidly supported by amputated residual patient bone, wherein the implant protrudes through overlying stump tissue to an external location for suitable attachment to a prosthetic limb or the like. A non-invasive electrical stimulation system provides a controlled electrical field strength and current density at the implant-bone interface for achieving rapid and secure osseointegrated implant fixation in a significantly reduced rehabilitation time.

The electrical stimulation system comprises at least one and preferably multiple electrodes (anodes) mounted topically or externally onto the patient in close proximity to the implant-bone interface, with an external portion of the implanted mounting post providing a second electrode (cathode) for direct stimulation of periprosthetic bone. A most preferred electrode configuration includes a pair of band-shaped electrodes (anodes) wrapped externally about the residual limb respectively in proximity with proximal and distal ends of the implant-bone interface. In a preferred form, the implant comprises a conductive base structure formed from titanium or titanium alloy or the like. In a most preferred form, the implant is surface-coated with a thin film of a highly conductive substance such as gold for enhanced electrical conductivity.

In use, the electrode or electrodes (anodes) are placed externally onto the patient in proximity with the implant-bone interface, and the electrodes (anodes and cathodes) are coupled to an electrical stimulation circuit including an adjustable power supply for providing a controlled electrical field strength of about 1-10 volts/centimeter (V/cm) at the implant-bone interface, and a current density up to about 2 milliamps/cm² at the implant-bone interface. These field strength and current density numbers are believed to provide relatively rapid and substantially full strength post-bone attachment in a short rehabilitation period, typically on the order of as little as a few weeks, substantially in the absence of undesired tissue heating and resultant tissue necrosis.

Other features and advantages of the present invention will become apparent from the following more detailed description, taken in connection with the accompanying drawing which illustrate, by way of example, the principals of the present invention.

### BRIEF DESCRIPTION OF THE DRAWINGS

The accompanying drawings illustrate the invention. In such drawings:

FIG. 1 is a schematic diagram illustrating a surgically implanted post-shaped bone anchored orthopedic implant extending from an interface with patient bone to an external position, in combination with an electrical stimulation system for providing a rapid and improved strength implant-bone attachment interface;

FIG. 2 is a schematic diagram similar to FIG. 1, and illustrating a modified electrical stimulation system including multiple electrodes (anodes) mounted on the patient's skin;

FIG. 3 is a somewhat schematic diagram depicting an electrical stimulation system including a single strip-shaped electrode (anode);

FIG. 4 is a somewhat schematic diagram similar to FIG. 3, and illustrating an electrical stimulation system including a pair of strip-shaped electrodes (anodes);

FIG. 5 is a somewhat schematic diagram similar to FIGS. 3 and 4, but showing an electrical stimulation system with a single band-shaped electrode (anode); and

FIG. 6 is a somewhat schematic diagram similar to FIGS. 3-5, but depicting a most preferred embodiment including a pair of band-shaped electrodes (anodes) respectively mounted onto the patient at positions spaced a short distance proximally and distally from the end of the associated amputated patient bone.

### DETAILED DESCRIPTION OF THE PREFERRED EMBODIMENTS

As shown in the exemplary drawings, an osseointegrated implant for an amputee includes a surgically implanted post-shaped bone anchored orthopedic implant referred to generally by the reference numeral 10. This implant 10 is surgically implanted for direct and rigid attachment to and support by the residual amputated patient bone 12. The bone anchored implant 10 extends percutaneously from the bone fixation site, i.e., the implant-bone interface, to an external position for convenient and suitable attachment (not shown) to a prosthetic limb or the like. In accordance with the invention, a non-invasive electrical stimulation system 14 is externally and post-surgically applied for significantly and rapidly enhancing osseointegration at the post-bone interface in order to achieve rapid and secure, substantially full strength implant fixation in a significantly reduced rehabilitation time.

The bone anchored implant 10 comprises a percutaneous implant device for use in subsequent connection to a prosthetic limb or the like, wherein the limb prosthesis is linked rigidly to patient bone to provide an improved and more natural mechanical loading to result in an improved and more natural patient ambulation and gait. However, connection of the bone anchored implant 10 in a secure and stable manner to the residual patient bone 12 by osseointegration is a relatively lengthy biological process taking from a few months to as long as about 2 years to achieve adequate implant-bone fixation for withstanding normal mechanical loads of the type encountered during normal walking movements. By use of controlled electrical stimulation in accordance with the present invention, osteoblast migration at the implant-bone interface is significantly increased, to result in substantially full strength osseointegrated implant-bone fixation in a dramatically shortened rehabilitation period.

While the invention is shown and described herein in connection with a leg amputee, wherein eventual mechanical loading of the bone anchored implant 10 is substantially maximized, persons skilled in the art will recognize and appreciate that the bone anchored implant 10 may be used with other amputated limbs, such as an arm.

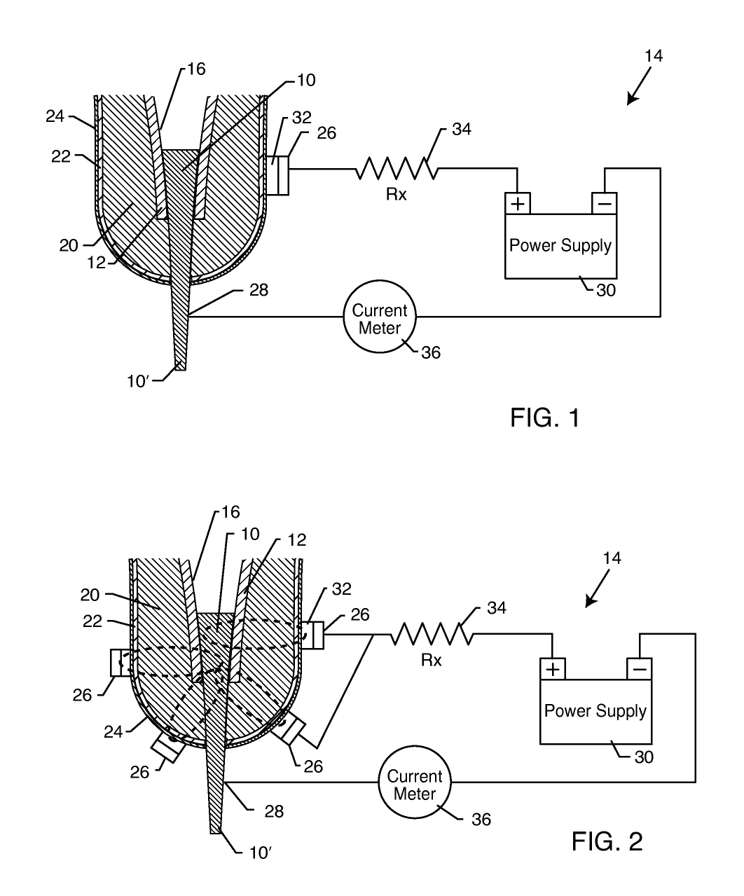

FIG. 1 illustrates the bone anchored implant 10 implanted surgically into an open-ended medullary canal 16 of an amputated long bone 12, such as an amputated femur in the case of a transfemoral amputee. Post-surgically, the amputation site is allowed to heal, resulting in soft tissue in the form of muscle tissue 20, fat or adipose tissue 22, and skin tissue 24 overlies the end of the amputated bone 12. However, the bone anchored implant 10 protrudes through this soft tissue, referred to commonly as stump tissue, for external exposure whereat an exposed end 10′ of the implant is available for subsequent fixation to a suitable prosthetic limb (not shown). One example of a suitable prosthetic limb is shown and described in US 2008/0288087, which is incorporated by reference herein.

In general, the bone anchored implant 10 is secured to the patient bone 12 by means of osseointegration, wherein the amputated patient bone 12 is allowed to grow and interknit securely with the implant 10 at an implant-bone interface defined typically by the endosteal wall lining the interior of the open-ended medullary canal 16 of the amputated patient bone 12 such as the illustrative femur. In this regard, as noted above, osseointegration is a relatively slow biological process which can take at least several months of post-surgical rehabilitation time for adequate implant-bone attachment to occur, especially in the case of a leg amputee wherein high mechanical loading of the implant-bone interface is anticipated during normal patient ambulation with a prosthesis attached to the implant 10. The rehabilitation time is further impacted by the length and condition of the residual amputated bone 12.

The electrical stimulation system 14 of the present invention comprises a non-invasive system applied post-surgically to the patient to achieve faster and substantially full strength osseointegrated attachment of the implant 10 to the patient bone 12, with a shortened rehabilitation time. As shown in FIG. 1, the electrical stimulation system 14 generally comprises at least one electrode (anode) 26 mounted externally or topically onto the patient skin tissue 24 at a position generally proximate to the implant-bone interface. In addition, the stimulation system 14 includes a second electrical lead 28 coupled to the external portion 10′ of the implant 10, whereby this external portion of the implant 10 comprises a second electrode (cathode). The electrodes (anode and cathode) 26, 28 are then coupled to a controllable or regulated power supply 30 which provides a controlled, preferably uniform and substantially homogeneous, electrical field strength and a limited or controlled current density at the implant-bone interface to promote rapid and secure attachment therebetween by osseointegration. In addition, using the osseointegrated implant 10 as a functional cathode for controlled electrical stimulation may serve to maintain and preserve host bone integrity in elderly patients with osseointegrated implants and prevent complications with osteopenia/osteoporosis and also prevent future revisions to osseointegrated implants.

FIG. 1 shows the electrical stimulation system 14 to include the electrode (anode) 26 preferably attached to the patient skin tissue 24 by means of a pad 32 adapted for high quality conductive skin contact. In this regard, the pad 32 comprises, in one preferred form, a foam pad of polyurethane foam or the like pre-soaked or pre-saturated with a saline solution or the like for improved electrical conductivity between the pad 32 and the adjacent skin tissue 24. Alternately, if desired, the pre-soaked pad 32 may be substituted by a suitable gel, such as an ultrasound transmission gel or the like.

In addition, FIG. 1 shows the controllable power supply 30 in the form of a direct current (dc) battery associated with a rheostat resistance (Rₓ) 34, wherein this adjustable component is set to provide the desired controlled electrical field strength and current density at the implant-bone interface to achieve relatively rapid post-bone attachment by osseointegration. A current meter 36 may also be included in the stimulation circuit for monitoring field strength and/or current density settings. Alternately, a digital multimeter (not shown) may be used in lieu of the adjustable rheostat and associated current meter.

More particularly, in a typical environment of operation, an electrical field strength at the implant-bone interface on the order of about 1 to about 10 volts/centimeter (V/cm) is desired, wherein the cm parameter comprises the length of the implant-bone attachment interface. Similarly, a current density at the implant-bone interface is desired up to about 2 milliamps/cm² of attachment surface area. Field strengths and/or current densities higher than these limits are believed to cause undesired localized heating of tissues and resultant tissue necrosis and/or patient discomfort. Maintaining these parameters within the indicated ranges is thus believed to dramatically shorten the overall implant-bone attachment time without undesired tissue heating. Solid state circuit components may be incorporated into the electrical stimulation system 14 for preventing these parameters from exceeding the stated limits.

For each amputee, the actual field strength and/or current density settings for the stimulation system 14 will vary according to anatomical deviations, in order to achieve the desired field strength and current density at the implant-bone interface. In this regard, the settings are determined empirically from a knowledge of the relative conductivities of various patient tissues, such as skin, muscle, fat (adipose), cortical bone, bone marrow, etc., in combination with the relative presence of these tissues in each individual patient. These empirically determined settings can be calculated for each individual patient using, e.g., mapping software available from www.scirun.org.

When sufficient implant-bone attachment strength is achieved, bone in-growth and bone on-growth are typically confirmed by the use of high resolution contact radiographs, and also by use of scanning electron microscopy (SEM) imaging.

FIG. 2 illustrates an alternative preferred embodiment of the invention, wherein multiple electrodes (anodes) 26 are coupled to the patient's skin tissue 24 at selected positions disposed in spaced relation about the amputated limb, with each electrode 26 providing an electrical field strength and current density targeted (as indicated by the dashed-line ovals) with different regions of the implant-bone interface. In this regard, the external mount positions of the multiple electrodes 26 are staggered both circumferentially and longitudinally relative to the post-bone interface. While four electrodes 26 are shown, it will be understood that different numbers of such electrodes may be provided in accordance with the physical geometry associated with a specific patient.

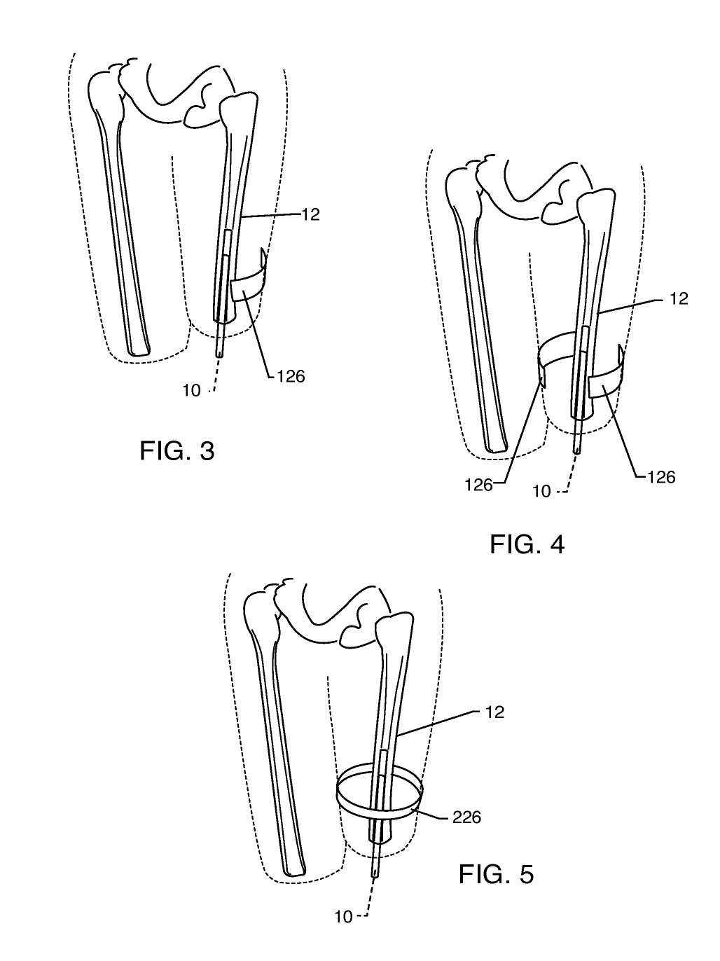

FIGS. 3-6 depict further schematic locations for different types of electrodes (anodes) 26. FIG. 3 shows a single band-type electrode 126 mounted at a laterally outboard side of an amputated limb, such as a leg. This band-type electrode 126 is elongated to provide electrical stimulation at the implant-bone interface corresponding with multiple pad-type electrodes 26 of the type shown in FIG. 2. FIG. 4 shows a pair of electrodes 126 mounted at both the laterally outboard and the medially inboard sides of the patient limb. FIG. 5 shows a single strap-type electrode 226 in the form of a full-circle or circumferential strap adapted for mounting onto the patient limb.

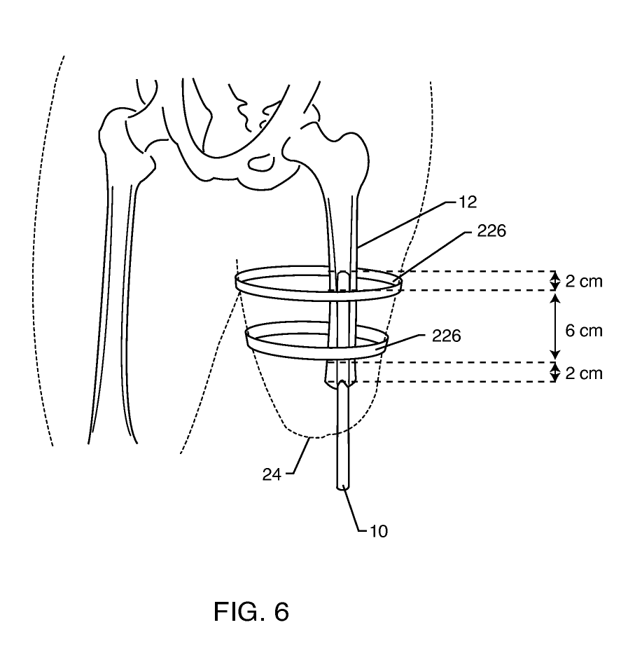

FIG. 6 illustrates a most preferred embodiment of the electrode (anode), wherein a pair of strap-type electrodes 226 (as illustrated in FIG. 5) are mounted in longitudinally spaced related about the patient limb. As shown, the two strap-type electrodes 226 are mounted respectively in relatively close proximity with the proximal and distal ends of the implant-bone interface. More particularly, as shown in a preferred arrangement, the two electrodes 226 are respectively mounted within about 2 cm inwardly from the associated proximal or distal end of the implant-bone interface, and are longitudinally separated by a spacing of about 6 cm. This arrangement of strap-type electrodes 226 has been found to be particularly effective in the rapid promotion of osseointegrated implant attachment to patient bone, with specific positioning varying according to the length of the implant-bone interface.

In accordance with one aspect of the invention, the bone anchored implant 10 is constructed from a strong and sturdy base structure having biocompatible properties and a mechanical strength suitable for withstanding mechanical loads during normal use, with a desirable relative low modulus of elasticity to prevent stress shielding. In this regard, a titanium-based implant material, such as titanium or titanium alloy is preferred. However, electrical conductivity of the implant 10 can be enhanced for improved and/or faster high strength osseointegrated attachment to patient bone, as by coating the base structure with a highly conductive film. In a preferred form, the highly conductive film comprises a gold or gold-based film.

More specifically, in a preferred form, the base structure of the implant 10 is film-coated as by ion beam deposition to adhere a first film layer of titanium for initial bonding, a second film layer of palladium for stabilizing the post surface, and a third film layer (thickness about 1,200 Angstroms) comprising a highly conductive gold coating. Such use of a highly conductive film on the implant 10 is believed to result in a substantial improvement in current flow to and through the implant-bone interface, for achieving the desired faster and stronger implant-bone attachment by osseointegration. In addition, the presence of the gold coating may also reduce the incidence of inflammation and/or foreign body response.

In addition to achieving a more rapid and secure implant fixation to patient bone, use of the electrical stimulation system 14 of the present invention is believed to prevent superficial and deep bacterial infections in the vicinity of the osseointegrated implant, and related osteolytic bone tissue degeneration. By preventing such post-surgical complications, subsequent corrective surgical procedures are also avoided.

Although various embodiments and alternatives have been described in detail for purposes of illustration, various further modifications may be made without departing from the scope and spirit of the invention. Accordingly, no limitation on the invention is intended by way of the foregoing description and accompanying drawings, except as set forth in the appended claims.

What is claimed is:

1. In combination:
a bone anchored implant coupled at an implant-bone interface to an amputated patient bone and extending percutaneously from the amputated patient bone to an external portion disposed at an external site for direct attachment to a prosthesis; and
a non-invasive electrical stimulation system for producing a controlled electrical field strength and current density at the implant-bone interface to achieve rapid and secure implant-bone fixation by osseointegration, said stimulation system including at least one electrode for topical mounting onto the patient's skin at a location generally proximate to the implant-bone interface, and a second electrode for mounting onto said external portion of said implant.

2. The combination of claim 1 wherein said at least one topically mounted electrode comprises an anode electrode, and further wherein said electrode mounted onto said implant comprises a cathode electrode.

3. The combination of claim 1 wherein said at least one topically mounted electrode comprises a plurality of electrodes.

4. The combination of claim 1 wherein said at least one topically mounted electrode comprises at least one strap-type electrode.

5. The combination of claim 1 wherein said at least one topically mounted electrode comprises a pair of strap-type electrodes mounted respectively in proximity with proximal and distal ends of said implant-bone interface.

6. The combination of claim 1 wherein said electrical stimulation system maintains a field strength at said implant-bone interface in the range of from about 1 to about 10 volts/centimeter, and further wherein said electrical stimulation system maintains a current density at said implant-bone interface of less than about 2 milliamps/centimeter².

7. The combination of claim 1 wherein said implant comprises a relatively strong and sturdy base structure having an external film coating applied thereto, said external film coating having highly conductive electrical properties.

8. The combination of claim 7 wherein said film coating comprises a gold film coating.

9. The combination of claim 8 wherein said base structure comprises a titanium-based material.

10. The combination of claim 1 wherein a portion of said implant is fitted into an open-ended medullary canal of the amputated patient bone to define said implant-bone interface, said implant extending from said medullary canal percutaneously through overlying stump tissue to said external portion.

11. In combination:
a bone anchored implant coupled at an implant-bone interface to an amputated patient bone and extending percutaneously from the amputated patient bone to an external portion disposed at an external site for direct attachment to a prosthesis;
said implant being formed from a titanium-based base structure having with a highly conductive film coating applied thereto; and
a non-invasive electrical stimulation system for producing a controlled electrical field strength at the implant-bone interface in the range of from about 1 to about 10 volts/centimeter and a controlled current density at the implant-bone interface of less than about 2 milliamps/centimeter² to achieve rapid and secure implant-bone fixation by osseointegration, said stimulation system including at least one electrode for topical mounting onto the patient's skin at a location generally proximate to the implant-bone interface, and a second electrode for mounting onto said external portion of said implant.

12. The combination of claim 11 wherein said film coating comprises a gold film coating.

13. The combination of claim 11 wherein said at least one electrode comprises a pair of strap-type electrodes mounted respectively in proximity with proximal and distal ends of said implant-bone interface.

14. A method for speeding osseointegrated fixation of a bone anchored implant to an amputated patient bone at an implant-bone interface, said implant extending from said implant-bone interface percutaneously to an external portion disposed at an external site for attachment to a prosthesis, said method comprising the steps of:
non-invasively electrically stimulating the implant-bone interface with a controlled electrical field strength and a controlled current density.

15. The method of claim 14 wherein said stimulating step comprising mounting at least one electrode topically on the patient's skin in proximity with said implant-bone interface, and mounting a second electrode onto said external portion of said implant.

16. The method of claim 15 wherein said step of mounting at least one electrode topically on the patient's skin comprises the step of mounting a pair of strap-type electrodes onto the patient's skin respectively in proximity with proximal and distal ends of said implant-bone interface.

17. The method of claim 14 wherein said controlled electrical field strength at said implant-bone interface is in the range of from about 1 to about 10 volts/centimeter, and further wherein said controlled current density at said implant-bone interface is less than about 2 milliamps/centimeter².

18. The method of claim 14 further comprising the step of surface-coating said post with a highly conductive surface film.

19. The method of claim 18 wherein said surface film comprises gold.
